(19) United States
(12) Patent Application Publication
(10) Pub. No.: US 2024/0366402 A1
(43) Pub. Date: Nov. 7, 2024

(54) SYSTEM, IMPLANT, AND METHODS FOR ARTHROPLASTY

(71) Applicant: Paragon 28, Inc., Englewood, CO (US)

(72) Inventors: Frank S. BONO, Lakeway, TX (US); Joseph DOGUÉ, Aurora, CO (US)

(73) Assignee: Paragon 28, Inc., Englewood, CO (US)

(21) Appl. No.: 18/776,417

(22) Filed: Jul. 18, 2024

Related U.S. Application Data
(63) Continuation of application No. PCT/US2023/060810, filed on Jan. 18, 2023.
(60) Provisional application No. 63/300,444, filed on Jan. 18, 2022.

(51) Int. Cl. A61F 2/46; A61F 2/42

(52) U.S. Cl. CPC A61F 2/4606; A61F 2002/4205; A61F 2002/4207; A61F 2002/4217

(57) ABSTRACT

An implant system having a tibial component, a talar component, and a calcaneal component. The implant system includes a first intermediate component disposed between the talar component and the tibial component and a second intermediate component disposed between the talar component and the calcaneal component. The talar component has a first articulating surface configured to engage with the first intermediate component and a second articulating surface configured to engage with the second intermediate component. The first articulating surface is disposed on a superior surface of the talar component and the second articulating surface is disposed on an interior surface of the talar component. Also disclosed is a subtalar arthroplasty system having an intermediate component and a calcaneal component. The intermediate component has a superior surface that is shaped to articulate with a talar component. The calcaneal component has an inferior surface that configured to promote coupling to a calcaneus bone.

### CROSS REFERENCE TO RELATED APPLICATION

This application is a bypass continuation of PCT Application No. PCT/US2023/060810, filed Jan. 18, 2023, and entitled “System, Implant, And Methods For Arthroplasty,” which claims benefit of priority of U.S. Provisional Patent Application No. 63/300,444, filed Jan. 18, 2022, and entitled “System, Implant, And Methods For Arthroplasty,” the disclosures of both are hereby incorporated herein by reference in their entirety.

### TECHNICAL FIELD

The present disclosure relates to arthroplasty systems which may include one or more of implants, instrumentation, and other devices as well as arthroplasty methodology. The present disclosure relates to podiatric and orthopedic arthroplasty systems to be implemented in arthroplasty procedures of the foot and/or ankle. More specifically, but not exclusively, the present disclosure relates to arthroplasty systems of the ankle joint and/or the subtalar joint.

### BACKGROUND OF THE INVENTION

Many currently available arthroplasty systems fail to consider various anatomical and physiological concerns of physicians and patients. In some instances, arthroplasty systems fail to account for anatomical structures adjacent an affected joint (e.g., musculoskeletal structures and kinematic properties) and, after implantation, may adversely affect said adjacent anatomical structures. Accordingly, arthroplasty systems are desired that account for not only the affected joint to be replaced by the arthroplasty system, but adjacent joints (and structures/properties thereof) as well.

### SUMMARY OF THE INVENTION

The present disclosure is directed toward surgical guides for implementation in conjunction with implants, instruments, and methods directed to arthroplasty procedures.

A first aspect of the present disclosure includes an implant system. The implant system includes a tibial implant component, a talar implant component, and a calcaneal implant component.

According to the first aspect of the present disclosure, the implant system includes a first intermediate component disposed between the talar implant component and the tibial implant component, and a second intermediate component disposed between the talar implant component and the calcaneal implant component.

According to the first aspect of the present disclosure, the implant system includes a first articulating surface configured to engage with the first intermediate component, and a second articulating surface configured to engage with the second intermediate component.

According to the first aspect of the present disclosure, the first articulating surface is disposed on a superior surface of the talar implant component, and the second articulating surface is disposed on an interior surface of the talar implant component.

According to the first aspect of the present disclosure, the tibial implant component includes a first engagement surface on a superior surface thereof configured to engage at least a portion of a tibia of the patient.

According to the first aspect of the present disclosure, the tibial implant component includes a second engagement surface on an inferior surface thereof configured to engage with a superior surface of the first intermediate component.

According to the first aspect of the present disclosure, the first articulating surface of the talar implant component is configured to engage with an inferior surface of the first intermediate component.

According to the first aspect of the present disclosure, the calcaneal implant component includes a first engagement surface on a superior surface thereof configured to engage with an inferior surface of the second intermediate implant.

According to the first aspect of the present disclosure, the calcaneal implant component includes a second engagement surface on an inferior portion thereof configured to engage with at least a portion of a calcaneus of the patient.

According to the first aspect of the present disclosure, the second articulating surface of the talar implant component is configured to engage with a superior surface of the second intermediate component.

A first aspect of the present disclosure includes a subtalar arthroplasty system. The subtalar arthroplasty system includes an intermediate component and a calcaneal implant component.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a rear view of an exemplary talar implant system, in accordance with the present disclosure;

FIG. 2 is a front view of the exemplary implant system of FIG. 1 for facilitating implantation of an implant system, in accordance with the present disclosure;

FIG. 3 is a first side view of the exemplary implant system of FIG. 1, in accordance with the present disclosure;

FIG. 4 is a second side view of the exemplary implant system of FIG. 1, in accordance with the present disclosure;

FIG. 5 is an elevated front perspective view of the exemplary implant system of FIG. 1, in accordance with the present disclosure;

FIG. 6 is an elevated rear perspective view of the exemplary implant system of FIG. 1, in accordance with the present disclosure;

FIG. 7 is a top view of a tibial component of the exemplary implant system of FIG. 1, in accordance with the present disclosure;

FIG. 8 is a bottom view of a calcaneal component of the exemplary implant system of FIG. 1, in accordance with the present disclosure;

FIG. 9 is a top view of a portion of the exemplary implant system of FIG. 1, in accordance with the present disclosure;

FIG. 10 is a front view of the portion of the exemplary implant system of FIG. 9, in accordance with the present disclosure;

FIG. 11 is a side view of the portion of the exemplary implant system of FIG. 9, in accordance with the present disclosure; and

FIG. 12 is a bottom view of the portion of the exemplary implant system of FIG. 9, in accordance with the present disclosure.

### DETAILED DESCRIPTION OF THE INVENTION

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

The instruments, implants, systems, assemblies, and related methods for maintaining, correcting, and/or resurfacing joint surfaces of the present disclosure may be similar to, such as include at least one feature or aspect of, the implants, systems, assemblies and related methods disclosed in disclosed U.S. Pat. No. 10,117,749, issued on Nov. 6, 2018 and entitled Subtalar Joint Implant; European Patent No. 3756626 issued on Dec. 30, 2020 and entitled Subtalar Joint Implant, European Patent Application No. 15770960.1A filed on Jul. 15, 2020 and entitled Subtalar Joint Implant; U.S. Provisional Patent Application No. 63/155,100 filed on Mar. 1, 2021 and entitled Methods for Performing Arthroplasty of the Subtalar Joint; U.S. Provisional Patent Application No. 63/167,965 filed on Mar. 30, 2021 and entitled Orthopedic Implants and Methods; International PCT Application No. PCT/US2019/29009, filed on Apr. 24, 2019, and entitled Implants and Methods of Use and Assembly; International PCT Application No. PCT/US2019/64741, filed on Dec. 12, 2019, and entitled Implant System and Methods of Use; International PCT Application No. PCT/US2019/66336, filed on Dec. 13, 2019, and entitled Patient Specific Instrumentation and Methods of Use; and/or International PCT Application No. PCT/US2019/66408, filed on Dec. 13, 2019, and entitled Joint Replacement Alignment Guides, System, and Methods of Use and Assembly; and/or International PCT Application No. PCT/US2019/66149 filed on Dec. 13, 2019, and entitled Alignment Instruments and Methods for Use in Total Ankle Replacement; and/or International PCT Application No. PCT/US2019/66393, filed on Dec. 13, 2019, entitled Joint Replacement Alignment Guides, Systems, and Methods of Use and Assembly; and/or U.S. Provisional Patent Application No. 62/898,615, filed on Sep. 11, 2019, entitled Resection Guides, Sweeping Reamers, and Methods for Use in Total Ankle Replacement; and/or International PCT Application No. PCT/US2019/66398, files on Dec. 13, 2019, entitled Distractors Having Attachable Paddles, Impaction Devices, and Methods for Use in Total Ankle Replacement; and/or International PCT Application No. PCT/US2019/65025, filed on Dec. 6, 2019, entitled Trial Insert Assembly; and/or U.S. Provisional Patent Application No. 62/899,460 filed Sep. 12, 2019, entitled Total Ankle Replacement Surgical Method; and/or International PCT Application No. PCT/US2019/66404 filed on Dec. 13, 2019, entitled Instruments, Guides, and Related Methods for Total Ankle Replacement; which are hereby incorporated herein by reference in their entireties. Similarly, the instruments, implants, systems, assemblies, and related methods for maintaining, correcting, and/or resurfacing joint surfaces of the present disclosure may include one or more instrument (e.g., one or more insertion and/or implantation instruments) disclosed in International PCT Application No. PCT/US2019/29009, filed on Apr. 24, 2019, and entitled Implants and Methods of Use and Assembly; International PCT Application No. PCT/US2019/64741, filed on Dec. 12, 2019, and entitled Implant System and Methods of Use; International PCT Application No. PCT/US2019/66336, filed on Dec. 13, 2019, and entitled Patient Specific Instrumentation and Methods of Use; and/or International PCT Application No. PCT/US2019/66408, filed on Dec. 13, 2019, and entitled Joint Replacement Alignment Guides, System, and Methods of Use and Assembly; and/or International PCT Application No. PCT/US2019/66149 filed on Dec. 13, 2019, and entitled Alignment Instruments and Methods for Use in Total Ankle Replacement; and/or International PCT Application No. PCT/US2019/66393, filed on Dec. 13, 2019, entitled Joint Replacement Alignment Guides, Systems, and Methods of Use and Assembly; and/or U.S. Provisional Patent Application No. 62/898,615, filed on Sep. 11, 2019, entitled Resection Guides, Sweeping Reamers, and Methods for Use in Total Ankle Replacement; and/or International PCT Application No. PCT/US2019/66398, files on Dec. 13, 2019, entitled Distractors Having Attachable Paddles, Impaction Devices, and Methods for Use in Total Ankle Replacement; and/or International PCT Application No. PCT/US2019/65025, filed on Dec. 6, 2019, entitled Trial Insert Assembly; and/or U.S. Provisional Patent Application No. 62/899,460 filed Sep. 12, 2019, entitled Total Ankle Replacement Surgical Method; and/or International PCT Application No. PCT/US2019/66404 filed on Dec. 13, 2019, entitled Instruments, Guides, and Related Methods for Total Ankle Replacement; and/or U.S. patent application Ser. No. 16/672,505 filed Nov. 3, 2019 and titled Talus Formational And Implantation Method; and/or International PCT Application No. PCT/US2021/046920 and titled Whole Talus Implant and Method; and/or International PCT Application No. PCT/US2021/047117 and titled Implant for Focal Talus Defects and Method; which are hereby incorporated herein by reference in their entireties.

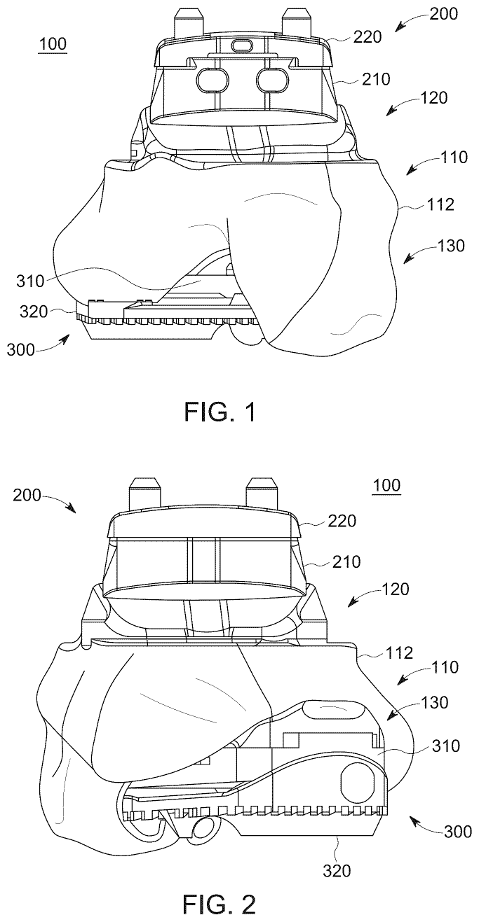

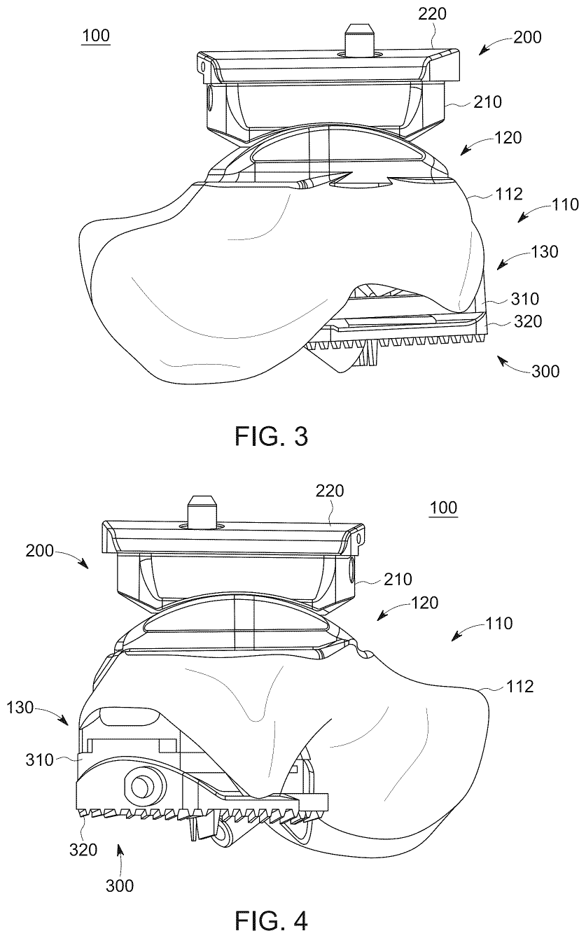

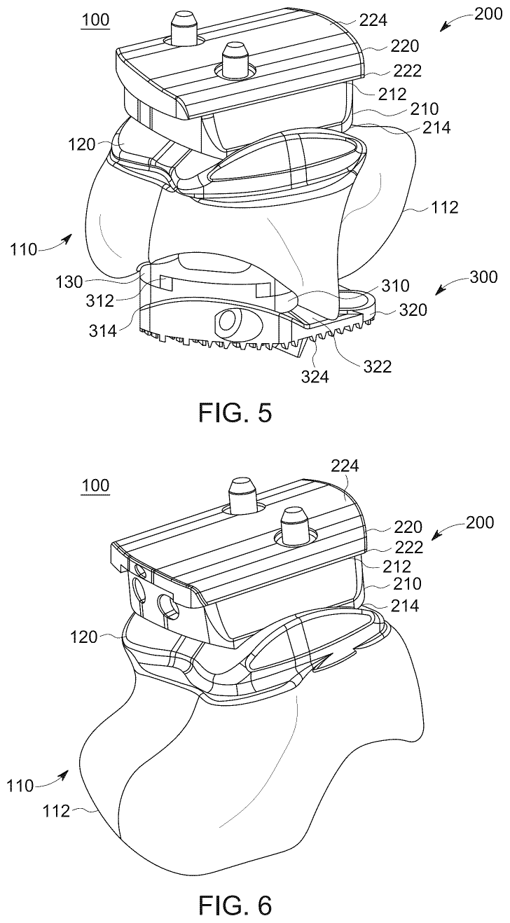

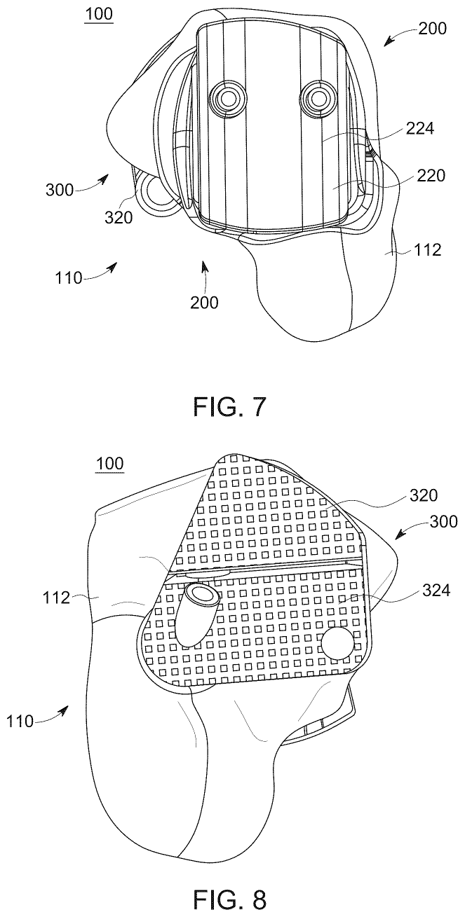

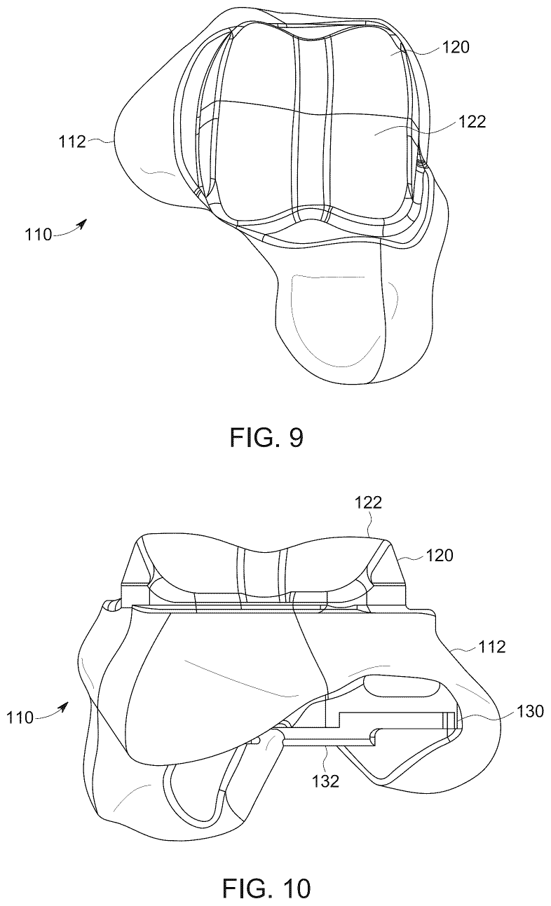

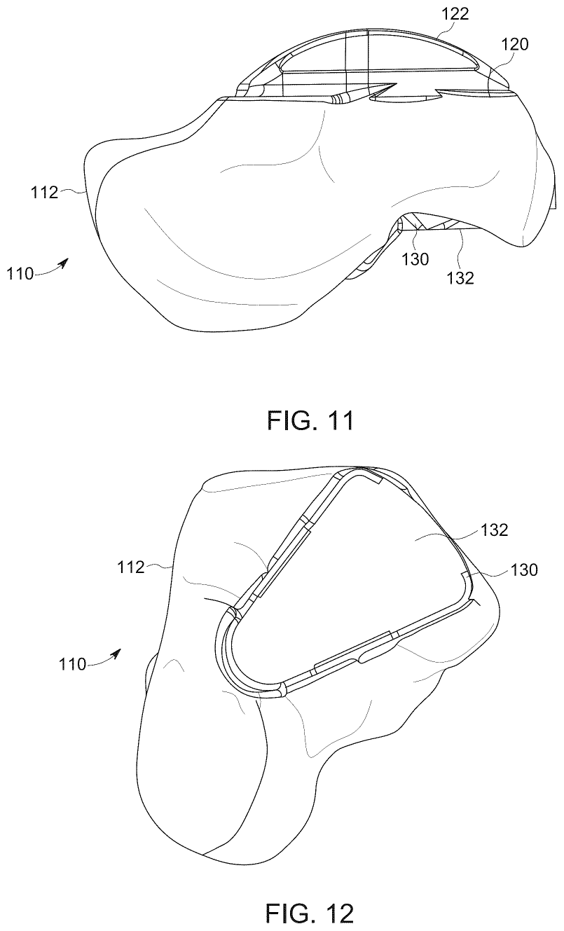

Referring to the drawings, wherein like reference numerals are used to indicate like or analogous components throughout the several views, and with particular reference to FIGS. 1-8, there is illustrated an exemplary embodiment of a dual-articulating talus replacement system 100 (which, for the sake of brevity, will be referenced herein as “talar implant system 100”). In some aspects, the talar implant system 100 may be configured to replace at least a portion of talus of a patient while articulating with both ankle and subtalar joint arthroplasty systems and/or components. It should be noted that although not shown in the Figures, the talar implant system 100 may be modified to alternate configurations. For example, the talar implant system 100 may be modified from the dual-articulating configuration as shown to a single-articulation implant configured to articulate with components of arthroplasty systems for ankle joints, subtalar joints, and/or other adjacent joints in the lower extremity. Further to the previous example, the talar implant system 100 may include a first articulating portion configured to articulate with a total ankle arthroplasty implant and a second articulating portion configured to articulate with a subtalar arthroplasty implant. In some aspects, the aforementioned total ankle arthroplasty implant and the subtalar arthroplasty implant with which the talar implant system 100 may articulate may not be otherwise compatible with one another except through the implementation of the talar implant system 100 shown as an intermediate component.

The talar implant system 100 is shown to include a talar implant 110 having a body 112. The body 112 may be configured to occupy at least a portion of the anatomy of a native talus of a patient. In some aspects, data may be collected (e.g., imaging, x-ray, etc.) in order to determine various geometric properties and/or volumes of a native talus of a patient such that the talar implant 110 (and portions thereof) may be produced (e.g., additively manufactured or generated through other production means) to have one or more properties the same as and/or similar to that of the native talus of the patient. In some aspects, the talar implant 110 may be additively manufactured or otherwise produced based on models developed from imaging data collected from a patient. For example, the talar implant 110 may be configured to have the same and/or similar geometric properties to that of the native talus of the patient so as to optimize the fit of the talar implant 110 and integration with surrounding anatomical structures. In some aspects, the talar implant 110 may include various features in addition to those shown and described herein. For example, the talar implant 110 may include one or more bone ingrowth surfaces (e.g., porous, surface texture, etc.) configured to contact anatomy adjacent the native talus and facilitate implantation and retention of the talar implant 110 within the patient, or may include one or more features configured to facilitate fixation (e.g., in a desired location adjacent patient anatomy, as a portion of an arthrodesis with joints adjacent the native talus, etc.). It should also be understood that the talar implant 110 as shown and described herein may also include one or more features, geometries, or other properties of talar implants shown and described in the pending patent applications mentioned previously and incorporated by reference herein in their entireties. Similarly, it should be understood that the talar implant 110 may also include one or more features of ankle arthroplasty systems and/or subtalar arthroplasty systems (or other arthroplasty systems for the ankle, subtalar joint, and/or other joints adjacent the talus) including but not limited to those identified previously and incorporated by reference herein in their entireties.

The talar implant 110 is further shown to include a first articulation 120 (e.g., a tibial articulation, a proximal articulation, a superior articulation, etc.) disposed on a superior (e.g., upper, proximal, tibial, etc.) portion of the talar implant 110 (and thus superior relative to the body 112), with said first articulation 120 having an articulating surface 122. The first articulation 120 and articulating surface 122 as shown may be the same as or similar to the articulation of the talar component of the Apex 3D™ total ankle arthroplasty system developed by Paragon 28, Inc. (or other similar systems) which is disclosed in the issued patents and pending patent applications listed previously and incorporated by reference herein in their entireties. In some aspects, the articulating surface 122 may include various radii of curvature (e.g., sagittal radii) over various areas of the articulating surface. For example, the articulating surface 122 may include a different radius over anterior-medial, anterior-lateral, posterior-medial, and posterior-lateral quadrants thereof. Further, in some aspects various quadrants may have greater/lesser radii than other adjacent quadrants (e.g., anterior-lateral quadrant has a greater radius of curvature than the anterior-medial quadrant, posterior-medial quadrant has a greater radius of curvature than the posterior-lateral, etc.). As shown, the first articulation 120 is disposed substantially superior to the body 112, although the arrangement of the first articulation 120 relative to the body 112 may be positioned alternatively based on the analysis of imaging or other data (e.g., imaging data from the patient).

The talar implant 110 is also shown to include a second articulation 130 (e.g., lower, distal, calcaneal, etc.) disposed on an inferior (e.g., lower, distal, calcaneal, etc.) portion of the talar implant 110 (and thus inferior relative to the body 112, and/or opposite the body 112 from the first articulation 120), with said second articulation 130 having an articulating surface 132. The second articulation 130 and articulating surface 132 as shown may be the same as or similar to the articulation of the one or more subtalar arthroplasty implants, for example that which is disclosed in the issued patents and pending patent applications listed previously and incorporated by reference herein in their entireties. In some aspects, the articulating surface 132 may include various features configured to engage (e.g., articulate with, releasably couple with, pivot about, etc.) corresponding features of one or more components of one or more subtalar arthroplasty systems (including but not limited to those incorporated by reference herein). For example, the articulating surface 132 may include lateral boundaries (e.g., an elongated protrusion(s)) configured to constrain movement of one or more subtalar arthroplasty components relative to the talar implant 110 (and/or vice-versa). As shown, the second articulation 130 is disposed substantially inferior to the body 112, although the arrangement of the second articulation 130 relative to the body 112 may be positioned alternatively based on analysis of imaging or other data (e.g., imaging data from the patient).

Referring now to FIGS. 1-7, the talar implant system 100 is shown to include an ankle arthroplasty system 200, according to an exemplary embodiment. The ankle arthroplasty system 200 is shown to include an intermediate component 210 configured to couple with a tibial component 220, where the tibial component 220 is configured for implantation in the distal tibia of a patient (e.g., a superior surface 222 of the tibial component 220 may include features/geometries configured to promote implantation in the distal tibia, for example bone ingrowth surfaces). The intermediate component 210 includes a superior surface 212 which may include one or more features configured to promote releasable coupling or other engagement with an inferior surface 224 of the tibial component 220. The intermediate component 210 is further shown to include an inferior surface 214 configured to interface with the articulating surface 122 of the first articulation 120 of the talar implant 110. In some aspects, the intermediate component may include one or more polymeric materials, for example UHMWPE and/or highly crosslinked polymeric materials infused with vitamin E. Further, in some aspects the intermediate component may be configured to articulate with either the first articulation 120 of the talar implant 110 or a superior surface of a talar component of a total ankle arthroplasty system, where the superior surface of both the talar implant 110 and said talar component have the same or similar geometries. It should be understood that the talar implant 100, the intermediate component 210 and the tibial component 220 may be configured to move (e.g., articulate, translate, etc.) relative to one or more of the other components of the ankle arthroplasty system 200.

Referring now to FIGS. 1-8, the talar implant system 100 is shown to include a subtalar arthroplasty system 300, according to an exemplary embodiment. It should be understood that the subtalar arthroplasty system 300 may be the same as and/or similar to other subtalar arthroplasty systems shown and described in patents and pending applications incorporated by reference herein. However, the subtalar arthroplasty system 300 may also include various components from various subtalar arthroplasty systems configured to articulate (interface, engage, releasably couple, etc.) with various components of the talar implant system 100. For example, the subtalar arthroplasty system 300 is shown to include an intermediate component 310 having a superior surface 312 configured to articulate with the articulating surface 132 of the second articulation 130 of the talar implant 110. In some aspects, the intermediate component 310 may have a size and/or geometry complimentary to at least a portion of the articulating surface 132, for example such that the intermediate component 310 is configured to be retained within a desired position relative to the talar implant 110 and is bound by one or more elongated protrusions extending from the articulating surface 132. The subtalar arthroplasty system 300 is further shown to include a calcaneal component 320 configured to be implanted adjacent to and/or coupled with a calcaneus of the patient. In some aspects, an inferior surface 324 of the calcaneal component 320 may include one or more features configured to promote coupling with the calcaneus of the patient (e.g., bone ingrowth surface, porosity, etc.). The calcaneal component 320 is also shown to include a superior surface 322 configured to articulate with an inferior surface of the intermediate component 310. In some aspects, the intermediate component 310 may include one or more features configured to engage with (e.g., releasably coupled, etc.) with one or more corresponding features of the calcaneal component 320. It should be understood that in some aspects, one or more of the talar implant 310, the intermediate component 310, and the calcaneal component 320 may be configured to articulate (e.g., move, pivot, etc.) with respect to one or more other components of the subtalar arthroplasty system 300.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

We claim:

1. An implant system, comprising:
a tibial implant component;
a calcaneal implant component; and
a talar implant component.

2. The implant system of claim 1, further comprising:
a first intermediate component disposed between the talar implant component and the tibial implant component; and
a second intermediate component disposed between the talar implant component and the calcaneal implant component.

3. The implant system of claim 2, wherein the talar implant component comprises:
a first articulating surface configured to engage with the first intermediate component; and
a second articulating surface configured to engage with the second intermediate component.

4. The implant system of claim 3, wherein the first articulating surface is disposed on a superior surface of the talar implant component and the second articulating surface is disposed on an interior surface of the talar implant component.

5. The implant system of claim 4, wherein the tibial implant component comprises a first engagement surface on a superior surface thereof configured to engage at least a portion of a tibia of a patient.

6. The implant system of claim 5, wherein the tibial implant component comprises a second engagement surface on an inferior surface thereof configured to engage with a superior surface of the first intermediate component.

7. The implant system of claim 6, wherein the first articulating surface of the talar implant component is configured to engage with an inferior surface of the first intermediate component.

8. The implant system of claim 4, wherein the calcaneal implant component comprises a first engagement surface on a superior surface thereof configured to engage with an inferior surface of the second intermediate implant.

9. The implant system of claim 8, wherein the calcaneal implant component comprises a second engagement surface on an inferior portion thereof configured to engage with at least a portion of a calcaneus of the patient.

10. The implant system of claim 8, wherein the second articulating surface of the talar implant component is configured to engage with a superior surface of the second intermediate component.

11. The implant system of claim 4, wherein the first articulating surface comprises a plurality of radii.

12. The implant system of claim 11, wherein the plurality of radii are different for an anterior-medial quadrant, an anterior-lateral quadrant, a posterior-medial quadrant and a posterior-lateral quadrant of the first articulating surface.

13. The implant system of claim 12, wherein the radius of curvature of the anterior-medial quadrant is lesser than the radius of curvature of the anterior-lateral quadrant and the radius of curvature of the posterior-medial quadrant is greater than the radius of curvature of the posterior-lateral quadrants of the first articulating surface.

14. The implant system of claim 10, wherein the second articulating surface is comprised of at least one elongated protrusion, wherein the at least one elongated protrusion is configured to constrain movement of a subtalar arthroplasty component.

15. A subtalar arthroplasty system, comprising:
an intermediate component; and
a calcaneal implant component.

16. The subtalar arthroplasty system of claim 15, wherein the intermediate component comprises a superior surface, wherein the superior surface is configured to articulate with an articulating surface of a talar implant component.

17. The subtalar arthroplasty system of claim 16, wherein the superior surface is sized to be complimentary to at least a portion of the articulating surface of the talar implant component.

18. The subtalar arthroplasty system of claim 15, wherein the calcaneal implant component comprises an inferior surface.

19. The subtalar arthroplasty system of claim 18, wherein the inferior surface of the calcaneal implant component comprises a plurality of surface features to promote coupling of the inferior surface to a calcaneus bone.

20. The subtalar arthroplasty system of claim 19, wherein at least one of the plurality of surface features is a post.
